(19) United States
(12) Patent Application Publication
(10) Pub. No.: US 2007/0224639 A1
(43) Pub. Date: Sep. 27, 2007

(54) Substrate for immobilizing biomolecules, biochip, and biosensor

(75) Inventors: Tomohiko Matsushita, Osaka (JP); Takeo Nishikawa, Kyoto (JP); Hideyuki Yamashita, Kyoto (JP); Masaaki Ikeda, Kyoto (JP); Shigeru Aoyama, Kyoto (JP); Tetsuichi Wazawa, Miyagi (JP); Hiroshi Sezaki, Tokyo (JP)

Correspondence Address: OSHA LIANG L.L.P., 1221 MCKINNEY STREET, SUITE 2800, HOUSTON, TX, 77010, US

(73) Assignees: OMRON Corporation, Kyoto-shi (JP); Osaka University, Osaka (JP)

(21) Appl. No.: 11/477,153

(22) Filed: Jun. 28, 2006

(30) Foreign Application Priority Data
Jun. 30, 2005 (JP) 2005-192746
May 15, 2006 (JP) 2006-135798

(51) Int. Cl. G01N 33/53; C12M 3/00

(52) U.S. Cl. 435/7.1; 435/7.5; 435/287.2; 977/902

(57) ABSTRACT

A substrate for immobilizing biomolecules comprises a chip substrate, a hydrophilic monolayer, and a lipid bilayer, and a biochip comprising the substrate for immobilizing biomolecules on which biomolecules are immobilized. The substrate for immobilizing biomolecules includes a transparent chip substrate, a metal layer provided on the chip substrate, a monolayer provided on the metal layer, and a lipid bilayer provided on the monolayer. The metal layer is composed of fine particles of Au, the monolayer is composed of self-assembled molecules represented by X—(CH₂)ₙ—OH (where X is a thiol group), and the lipid bilayer is composed of self-assembled phospholipids. The monolayer and the lipid bilayer are relatively flexibly bound together via hydrogen bonds. In the biochip, a receptor is immobilized on the lipid bilayer via a biorecognition molecule.

### BACKGROUND OF THE RELATED ART

1. Field of the Invention

The present invention relates to a substrate for immobilizing biomolecules, a biochip, and a biosensor.

2. Description of the Related Art

Biosensing

Application of biochips or quantum chips, obtained by two-dimensionally arranging biomolecules on a chip substrate, to medical, environmental, electronics, and other various fields has been explored. Particularly, in medical and diagnostic fields and in the field of research on mechanisms of living organisms, protein chips, obtained by two-dimensionally arranging many protein molecules on a chip substrate, are required for various purposes such as disease diagnosis, physical examination, person authentication, analysis of system of living organisms, and the like.

For example, in order to understand the system of living organisms, it is necessary to clarify the network of interaction between protein molecules expressed in cells and the time fluctuation of the network. Therefore, there is a strong demand for construction of protein chips enabling high throughput analysis of the interaction between expressed proteins.

A protein chip is formed by two-dimensionally arranging and immobilizing various kinds of probes (proteins) on a chip substrate. When a sample is brought into contact with such a protein chip, only a specific target (protein) contained in the sample, which is determined according to the characteristics of a probe, binds to a probe. Therefore, it is possible to identify the kind of the target protein and to clarify the expression and interaction of proteins by detecting the characteristic change of the probe caused by binding with the target, converting it to optical or electrical signals, and reading the signals to determine the presence or absence of characteristic change of the probe or the amount of the target.

For example, in a case where a sample such as blood is brought into contact with a protein chip obtained by two-dimensionally immobilizing an antibody on a chip substrate, only a certain antigen (e.g., a certain virus such as Bacillus anthracis or smallpox) is reacted with the antibody and adsorbed to the protein chip, thereby allowing the detection of the presence or absence of the certain antigen. Further, it is possible to measure the amount of the antigen adsorbed to the antibody immobilized on the protein chip or the amount of the antigen removed from the sample. In this way, the presence or absence of infection caused by a certain bacterium or the extent of disease is determined.

Further, protein chips are expected to be useful for development of specific agents for incurable diseases, development of drugs with no side-effects, and achievement of preventive medicine.

It is to be noted that examples of such a protein chip to be used for biosensing include: (1) protein chips obtained by immobilizing an antibody, a pseudo-antibody, an aptamer, or a phage display on a substrate; (2) protein chips obtained by immobilizing a protein expressed from CDNA on a substrate; and (3) protein chips obtained by immobilizing a protein purified from cells or tissues on a substrate.

Lipid Bilayer

In order to immobilize an antibody on a chip substrate of such a biochip (protein chip) described above, it is necessary to first form a lipid bilayer on the surface of the chip substrate and then immobilize a protein such as an antibody on the lipid bilayer. A lipid bilayer is a basic structure of a biological membrane, and the basic skeleton of the biological membrane can be obtained by embedding or binding proteins in or to the lipid bilayer. Therefore, proteins immobilized on the surface of a lipid bilayer artificially formed on a chip substrate or proteins embedded in such a lipid bilayer can express their intrinsic physiological functions. Based on the fact, various methods for artificially forming a lipid bilayer on the surface of a chip substrate have been proposed.

One conventional biosensor has a recording electrode provided in a chip substrate (Teflon block). On the recording electrode, a lipid bilayer is provided in such a manner that there exists a bulk aqueous layer between the electrode and the lipid bilayer. Further, a reference electrode is provided above the lipid bilayer. The lipid bilayer is attached to the recording electrode via bridging anchoring molecules composed of a hydrophilic spacer molecule.

As such a bridging anchoring molecule, phosphatidylethanolamine linked to a polyoxyalkylene chain terminated by a thiol or thioether residue is used. Alternatively, PE-NH—(CH₂—CH₂—O)n-CH₂—CH₂—SH (n is about 7 to 24, PE-NH represents a residue of phosphatidylethanolamine) may be used as a bridging anchoring molecule. The bridging anchoring molecules are attached to the surface of the recording electrode via the terminal thiol or thioether residues thereof, and the bridging anchoring molecules are covalently bound to the lipid bilayer.

In another conventional biosensor, an Au layer is provided on the surface of a chip substrate, a lipid bilayer is provided on the chip substrate via spacer molecules, and a receptor is embedded in the lipid bilayer

As such a spacer molecule, a molecule containing a peptide (more specifically, a molecule composed of 1 molecule of ethanolamine, an oligopeptide in helix or pleated-sheet structure formed from 4 to 20 C₂-C₁₀-α amino acids, and a reactive group which enters into a chemical or physicochemical bond with the chip substrate) is used. The ethanolamine of the spacer molecule is bound to a phosphoric group of the lipid bilayer by a covalent bond (ester bond).

As described above, in these conventional biosensors, the lipid bilayer and the bridging anchoring molecules or the spacer molecules (molecules containing a peptide) are strongly bound together by a covalent bond. That is, the lipid bilayer is directly immobilized on the chip substrate via the bridging anchoring molecules or the spacer molecules, which impairs flexibility of the lipid bilayer. Therefore, there is a fear that such a lipid bilayer of the conventional biosensor is deactivated, which further causes a drawback that the lifetime of the lipid bilayer is shortened.

Generally, biomolecules act in fluid media. However, in a case where bridging anchoring molecules or spacer molecules are used for immobilizing a lipid bilayer on a chip substrate, the lipid bilayer and biomolecules bound to the lipid bilayer lack flowability. Therefore, there is a fear that it is impossible to observe intrinsic functions or activities of the biomolecules because they are limited. Further, since a general chip substrate includes an expensive Au layer, it is reused. However, in a case where a lipid bilayer is immobilized on a chip substrate via bridging anchoring molecules or spacer molecules, the lipid bilayer is strongly bound to the chip substrate, and therefore it is difficult to reuse the chip substrate.

The lipid bilayer of the conventional biosensor is formed by the following method. First, ethanolamine molecules are bound to hydrophilic parts of phospholipids, and then 4 to 20 α-amino acids are bound to a nitrogen atom of each of the ethanolamine molecules to form spacer molecules and a monolayer of phospholipids. Thereafter, a diphosphatidyl compound containing the spacer molecules is immobilized on a chip substrate via the HS regions of the spacer molecules. Then, a liposome solution is added to fuse lipid monolayers together to form a lipid bilayer on the chip substrate.

However, such a lipid bilayer forming method is not efficient because the step of forming spacer molecules and a monolayer of phospholipids and the step of forming a lipid bilayer both require a lot of effort.

In the case of still another conventional biosensor, a lipid bilayer is formed on a chip substrate via hydrophilic peptide molecules having a hydroxyl group, and the lipid bilayer is hydrogen-bonded to hydroxyl groups of the peptide molecules. The peptide molecule is an oligopeptide having one or more reactive groups such as —SH, —OH, —COOH, and —NH for linkage.

In this conventional biosensor, since the lipid bilayer is hydrogen-bonded to the peptide molecules and is relatively weakly anchored to the chip substrate via the peptide molecules, deactivation of biomolecules immobilized on the lipid bilayer can be prevented and membrane proteins can also be immobilized on the lipid bilayer. Further, since the biosensor uses a conductive peptide as means for binding the lipid bilayer to the chip substrate, electrical signals can be transmitted through the peptide molecules, thereby allowing the detection of change in the biomolecules by measuring the electrical change of the biosensor.

However, it is impossible for the biosensor to provide the peptide molecules on the chip substrate at high density due to the structure of the peptide molecule. Therefore, it is difficult to firmly anchor the lipid bilayer to the chip substrate, and therefore separation of the lipid bilayer is likely to occur. Further, since the peptide molecule is poor in stability and is soft, the lipid bilayer anchored to the chip substrate via the peptide molecules is likely to change with the lapse of time.

Furthermore, in the case of such a biosensor using peptide molecules, it is difficult to control the thickness of the layer of peptide molecules to be uniform, which also makes it difficult to optionally set the distance between an electrode formed in the chip substrate and the lipid bilayer. Therefore, when biomolecules immobilized on the lipid bilayer are analyzed by optical sensing, especially by SPR (surface plasmon resonance), analytical accuracy is not constant. As described above, since it is difficult to make the thickness of the layer of peptide molecules uniform, analysis of biomolecules by SPR results in poor analytical accuracy due to many noises.

The lipid bilayer of this conventional biosensor is formed by the following method. First, peptide molecues (R-A-B-C-D-E-OH) are synthesized, and then the R groups thereof are bound to an electrode to form a monolayer of the peptide molecules. Then, liposomes composed of phosphatidylcholine or phospholipid containing phosphatidic acid-NH₂ group are fused to the peptide molecules to immobilize a lipid bilayer on the electrode. However, such a lipid bilayer forming method is not efficient because the step of forming a monolayer of peptide molecues and the step of forming a lipid bilayer both require a lot of effort.

### SUMMARY

Embodiments of the present invention provide a novel substrate for immobilizing biomolecules which comprises a chip substrate, a hydrophilic monolayer, and a lipid bilayer, and a biochip comprising the substrate for immobilizing biomolecules on which biomolecules are immobilized.

In accordance with one aspect of the present invention, a substrate for immobilizing biomolecules comprises a substrate; anchoring molecules provided on the substrate; and a lipid bilayer provided on the anchoring molecules, wherein the anchoring molecules are represented by X—(CH₂)n-OH (where X is a thiol group) and form a layer; and the lipid bilayer is anchored to the substrate via hydrogen bonds existing between the lipid bilayer and the anchoring molecules.

In accordance with another aspect of the present invention, a biochip comprises a substrate, anchoring molecules provided on the substrate, a lipid bilayer provided on the anchoring molecules; a biorecognition molecule immobilized on the lipid bilayer; and a receptor immobilized on the biorecognition molecule, wherein the anchoring molecules are represented by X—(CH₂)n-OH (where X is a thiol group) and form a layer; the lipid bilayer is anchored to the substrate via hydrogen bonds existing between the lipid bilayer and the anchoring molecules; and the receptor specifically binds to a specific protein (ligand).

In accordance with another aspect of the present invention, a biosensor comprises a biochip; and a measuring apparatus, wherein the biochip comprises a substrate; anchoring molecules provided on the substrate; a lipid bilayer provided on the anchoring molecules; a biorecognition molecule immobilized on the lipid bilayer; and a receptor immobilized on the biorecognition molecule, wherein the anchoring molecules are represented by X—(CH₂)n-OH (where X is a thiol group) and form a layer; the lipid bilayer is anchored to the substrate via hydrogen bonds between the lipid bilayer and the anchoring molecules; and the receptor specifically binds to a specific protein (ligand), and wherein the measuring apparatus detects a reaction state such as the presence or absence of an analyte as a test object, the amount of the analyte, or the binding specificity of the analyte.

In accordance with another aspect of the present invention, a method for forming a substrate to which a lipid bilayer is anchored comprises the steps of: forming a layer by arranging anchoring molecules represented by X—(CH₂)n-OH (where X is a thiol group) on the surface of a substrate by self-assembly; and forming on the layer formed by the anchoring molecules, a lipid bilayer by lipid self-assembly and anchoring the lipid bilayer to the substrate via hydrogen bonds existing between the lipid bilayer and the anchoring molecules.

It is to be noted that the components in the embodiments of the present invention described above can be combined as freely as possible.

### BRIEF DESCRIPTION OF THE DRAWINGS

FIG. 1 shows a schematic view of a configuration of a biochip according to an embodiment of the present invention;

FIG. 2 shows a graph illustrating the relationship between the number of methylene groups contained in a monolayer and the thickness of the monolayer according to an embodiment of the present invention;

FIGS. 3A to 3F show illustrations for explaining the process of forming a monolayer on the surface of a chip substrate according to an embodiment of the present invention;

FIG. 4 shows a schematic diagram of a phospholipid vesicle according to an embodiment of the present invention;

FIGS. 5A to 5D show illustrations for explaining the process of preparing a phospholipid vesicle according to an embodiment of the present invention;

FIGS. 6A and 6B show illustrations for explaining the process of forming a lipid bilayer by applying the phospholipid vesicles onto the chip substrate according to an embodiment of the present invention;

FIG. 7 shows a schematic view of a structure of a biosensor according to an embodiment of the present invention;

FIG. 8 shows a graph illustrating a change in reflectivity measured with the biosensor at various incident angles of incident light according to an embodiment of the present invention;

FIG. 9 shows a schematic view of a model used for simulation according to an embodiment of the present invention;

FIG. 10 shows a table for illustrating changes in resonance angle and reflectivity at the time when the thickness of the monolayer was changed according to an embodiment of the present invention;

FIG. 11 shows a graph obtained by plotting the values listed in FIG. 10 to illustrate a change in reflectivity according to an embodiment of the present invention;

FIG. 12 shows a table for illustrating changes in resonance angle and reflectivity at the time when the thickness of the lipid bilayer was changed according to an embodiment of the present invention;

FIG. 13 shows a graph obtained by plotting the values listed in FIG. 12 to illustrate a change in reflectivity according to an embodiment of the present invention;

FIG. 14 shows a table for illustrating changes in resonance angle and reflectivity at the time when the thickness of a metal layer was changed according to an embodiment of the present invention; and

FIG. 15 shows a graph obtained by plotting the values listed in FIG. 14 to illustrate a change in reflectivity according to an embodiment of the present invention.

### DETAILED DESCRIPTION

Hereinbelow, one of the embodiments of the present invention will be described in detail with reference to the accompanying drawings.

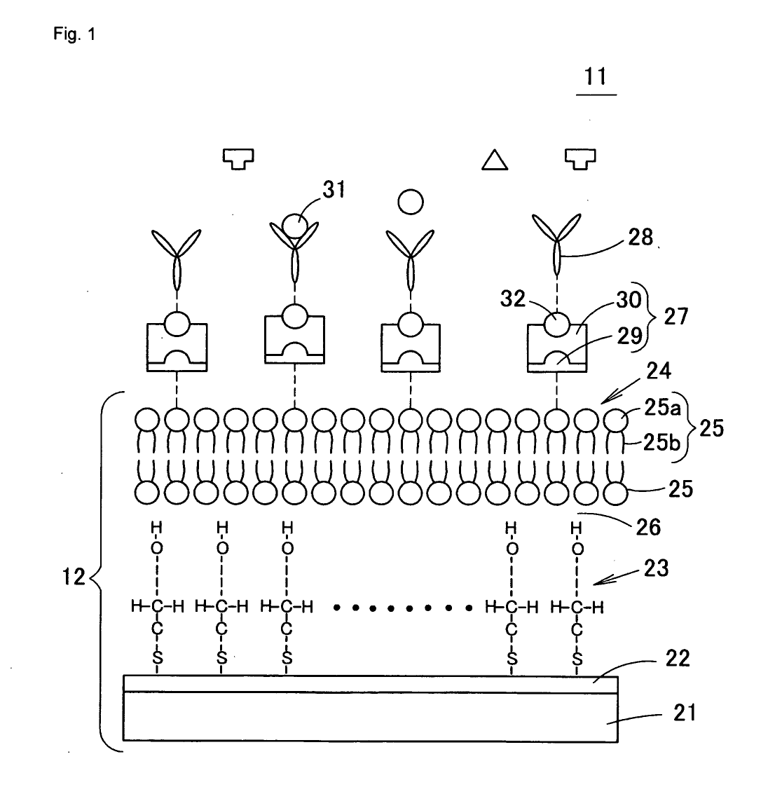

FIG. 1 shows a schematic view of a configuration of a biochip 11 (that is, a substrate for immobilizing biomolecules 12 on which a receptor is immobilized). As will be described later in detail, the substrate for immobilizing biomolecules 12 includes a chip substrate 21, a metal layer 22 provided on the surface of the chip substrate 21, a hydrophilic monolayer 23 provided on the metal layer 22, and a lipid bilayer 24 anchored to the chip substrate 21 via the monolayer 23. The biochip 11 is formed by immobilizing a biorecognition molecule 27 on the lipid bilayer 24 of the substrate for immobilizing biomolecules 12 and then further immobilizing a receptor 28 on the biorecognition molecule 27.

The chip substrate 21 is formed from a sheet of a translucent material such as glass or quartz. On the upper surface of the chip substrate 21, a plurality of metal fine particles are immobilized to form the metal layer 22.

The metal fine particles forming the metal layer 22 are nano-sized inorganic metal fine particles, such as Au or Ag, having a diameter of several tens of nanometers (particularly, a diameter of 40 to 50 nm). These metal fine particles immobilized on the chip substrate 21 hardly agglomerate, that is, they are separated from each other on the chip substrate 21. The metal fine particles are not necessarily arranged regularly. For example, they may be dispersed in a random fashion. In several embodiments, the interval between adjacent metal fine particles (that is, the distance between the surfaces of the metal fine particles at the centers of the adjacent metal fine particles, which is the shortest distance between the surfaces of adjacent metal fine particles) is two times or more but 4 times or less the diameter of the metal fine particle. For example, the density of metal fine particle of about 370 particles/μ² corresponds to a coverage factor of about 0.17.

The hydrophilic monolayer 23 provided on the metal layer 22 is composed of self-assembled molecules, and the lipid bilayer 24 is anchored to the monolayer 23. More specifically, the monolayer 23 is formed by self-assembly of molecules (spacer molecules) represented by X—(CH₂)ₙ—OH (where X is a thiol group), and the thiol group X of each of the molecules is immobilized on the metal layer 22 (or on the chip substrate 21). Such a molecule constituting the hydrophilic monolayer 23 can also be represented by HS(CH₂)ₙOH (thioalkanol). In several embodiments, the thickness of the monolayer 23 is 1 nm or less. Further, the monolayer 23 is kept as thin as possible.

The lipid bilayer 24 is composed of two adjacent layers of amphiphilic phospholipids 25 arranged in such a manner that hydrophobic parts 25b of the phospholipids 25 are faced to each other. The lipid bilyaer 24 is bound via hydrogen bonds to the monolayer 23, thereby enabling the lipid bilayer 24 to be anchored to the surface of the chip substrate 21. In this regard, it is to be noted that the lipid bilayer 24 is not directly hydrogen-bonded to the monolayer 23, but the lipid bilayer 24 and the monolayer 23 are bound together via water molecules which are present as a medium 26 between the lipid bilayer 24 and the monolayer 23. More specifically, the monolayer 23 is immobilized on the chip substrate 21 by attaching thiol groups X thereof to the metal layer 22, hydroxyl groups (OH) of the monolayer 23 are hydrogen-bonded to water molecules, and the water molecules are hydrogen-bonded to hydrophilic parts of the lipid bilayer 24 (that is, to hydrophilic parts 25a of the phospholipids 25), thereby enabling the lipid bilayer 24 to be anchored via the monolayer 23 to the chip substrate 21. In several embodiments, the thickness of the lipid bilayer 24 is 5 to 10 nm. Further, the lipid bilayer 24 is kept as thin as possible.

As described above, since the lipid bilayer 24 and the monolayer 23 are relatively weakly bound via hydrogen bonds, the lipid bilayer 24 is flexibly anchored to the chip substrate 21. Therefore, the lipid bilayer 24 of the biochip 11 is hard to be deactivated, thereby increasing the lifetime of the lipid bilayer 24. Further, such flexible anchoring of the lipid bilayer 24 to the chip substrate 21 makes it hard to inhibit flowability of the lipid bilayer or biomolecules bound to the lipid bilayer, thereby allowing the observation of intrinsic functions or activities of the biomolecules.

In several embodiments, the molecular density of the monolayer 23 is 1 molecule/nm² or more. On page 7749 of the article entitled “pH-Dependent Behavior of Surface-immobilized Artificial Leucine Zipper Protains” (Molly M. Stevens et al.; Langmuir 2004, 20, 7747-7752, American Chemical Society), it is described that peptides were immobilized on the Au layer at a density of 708 ng/cm². This value corresponds to a molecular density of 0.5 molecules/nm², which can be considered as the maximum molecular density of peptides that can be formed on the Au layer. On the other hand, according to the article entitled “Self-assembled membrane of thioalkane alcohol” (Deboirs, L. H. & Nuzzo, R. G. (1992) Annu. Rev. Phys. Chem. 43: 437), the density of a typical thioalkane alcohol, HS—(CH₂)₁₁—OH (Mw=204.37) is 157 ng/cm². This value corresponds to a molecular density of 4.8 molecules/nm².

In the case of the hydrophilic monolayer 23, molecules can be arranged at a higher density, especially at a density of 1 molecule/nm² or more, as compared to the conventional method using peptide molecules. Therefore, the biochip 11 can have the monolayer 23 having a high molecular density. By increasing the molecular density of the monolayer 23, it is possible to increase the bonding strength of the lipid bilayer 24 to the metal layer 22, thereby enabling the lipid bilayer 24 to be stabilized and suppressing a change with time in the lipid bilayer 24. Further, by controlling the molecular density of the monolayer 23, it is possible to modulate the bonding strength of the lipid bilayer 24 to the metal layer 22.

The article entitled “Peptide-derived Self-assembled Monolayers: Adsorption of N-stearoyl L-Cysteine Methyl Ester on Gold” (Susan L. Dawson and David A. Tirrell: Journal of Molecular Recognition, Vol., 10, 18-25 (1997)) reports that peptide molecules are arranged in a disorderly manner in the self-assembled monolayer of peptide on the Au layer. Therefore, in the case of such a conventional peptide monolayer, it is difficult to make the thickness thereof uniform.

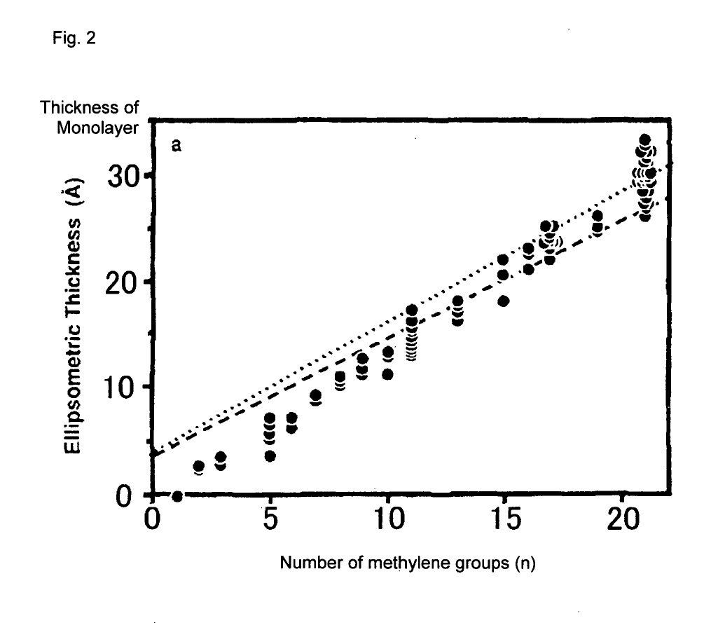

On the other hand, in the case of the monolayer 23, it is possible to make the thickness thereof uniform. Further, it is also possible to control the thickness thereof with angstrom (Å) accuracy. FIG. 2 is a graph reprinted from the article entitled “Formation of Monolayer Films by the Spontaneous Assembly of Organic Thiols from Solution onto Gold” (Collin D. Bain et al.: J. Am. Chem. Soc. 1989, 111, 321-335), which shows the thickness of a monolayer, obtained by chemical adsorption of HS(CH₂)ₙOH₃ to an Au thin layer, experimentally measured by an ellipsometer. In FIG. 2, the horizontal axis represents the number (n) of methylene groups of the monolayer, and the vertical axis represents the thickness of the monolayer. As can be seen from FIG. 2, angstrom-scale linearity is recognized between the number (n) of methylene groups and the thickness of the monolayer. Therefore, in the case of the biochip 11, by controlling the number (n) of methylene groups of X—(CH₂)ₙ—OH constituting the monolayer 23, it is possible to obtain a monolayer 23 having a uniform thickness and to optionally adjust the thickness of the monolayer 23.

The biorecognition molecule 27 immobilized on the lipid bilayer 24 is composed of biotin 29 and avidin 30. The biotin 29 is immobilized on the lipid bilayer, and the avidin 30 is bound to the biotin 29. In a case where a lipid bilayer composed of phospholipids labeled with biotin is used, avidin can be directly immobilized on the lipid bilayer.

As the receptor 28, an antibody which specifically binds to a specific analyte 31 (protein) is selected, and the receptor 28 is labeled with biotin. A biotin part 32 of the receptor 28 is bound to the avidin 30 of the biorecognition molecule 27. In this way, the receptor 28 is immobilized on the biorecognition molecule 27.

As described above, since the thickness of the monolayer 23 of the biochip 11 can be made uniform, the thickness of the lipid bilayer 24 formed on the monolayer 23 can also be made uniform. This makes it easy to orient the biorecognition molecule 27 and the receptor 28 in an orderly manner on the lipid bilayer 24 so that the binding site of the receptor 28 can be exposed upward. As a result, a non-specific analyte is prevented from being adsorbed to the biorecognition molecule 27 or the receptor 28, thereby improving analytical accuracy and reliability of the biochip 11.

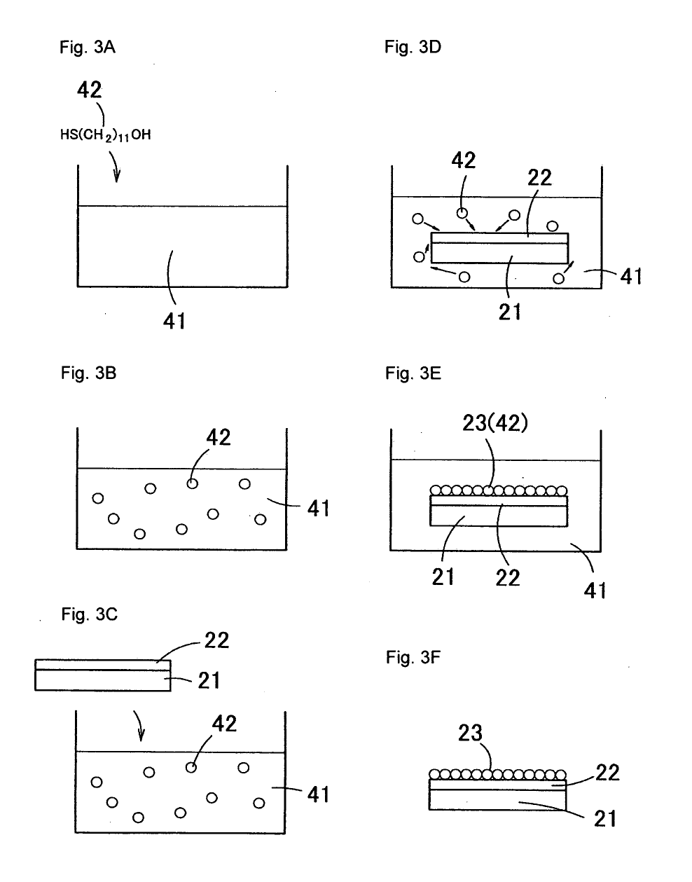

Next, an example of a method for producing a biochip 11 will be described with reference to FIGS. 3 to 6. First, as shown in FIG. 3A, thioalkanol 42 (HS(CH₂)₁₁OH) is added to a 100% ethanol solution 41. Then, as shown in FIG. 3B, the thioalkanol 42 is dissolved in the ethanol solution 41.

As shown in FIG. 3C, a chip substrate 21 whose one surface is covered with a metal layer 22 (that is, with an Au thin layer having a thickness of 40 to 50 nm) is immersed in the ethanol solution 41 for 1 hour. When the chip substrate 21 is immersed in the ethanol solution 41, the thioalkanol 42 dissolved in the ethanol solution 41 is deposited on the surface of the metal layer 22 and self-assembled as shown in FIG. 3D. Finally, as shown in FIG. 3E, a monolayer 23 composed of the thioalkanol 42 is formed on the metal layer 22.

Then, the chip substrate 21 is taken out of the ethanol solution 41, rinsed and dried. In this way, as shown in FIG. 3F, a target monolayer 23 is formed on the chip substrate 21. It is known that in the thus obtained monolayer 23, the thiol group of each of the thioalkanol molecules 42 is immobilized on the metal layer 22, and the thioalkanol molecules 42 are arranged parallel to each other and are tilted at several tens of degrees toward the surface of the metal layer 22.

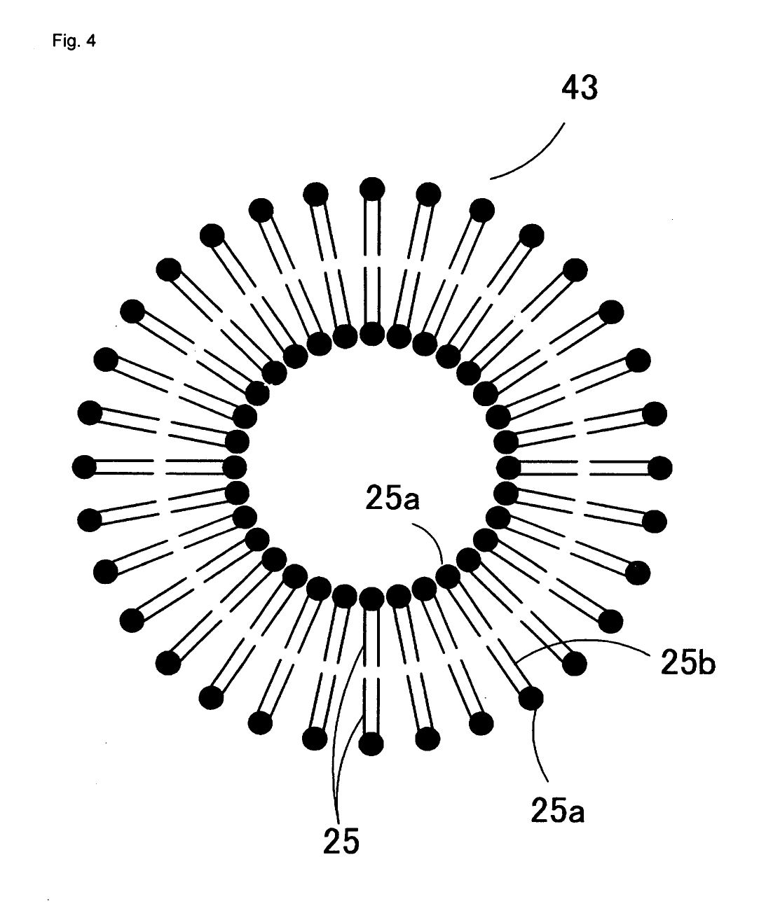

Then, phospholipid vesicles 43 are prepared. As shown in FIG. 4, a vesicle is a closed sphere formed from a lipid bilayer having a structure in which hydrophobic parts of phospholipids are faced to each other so that hydrophilic parts thereof can come into contact with an aqueous solution layer.

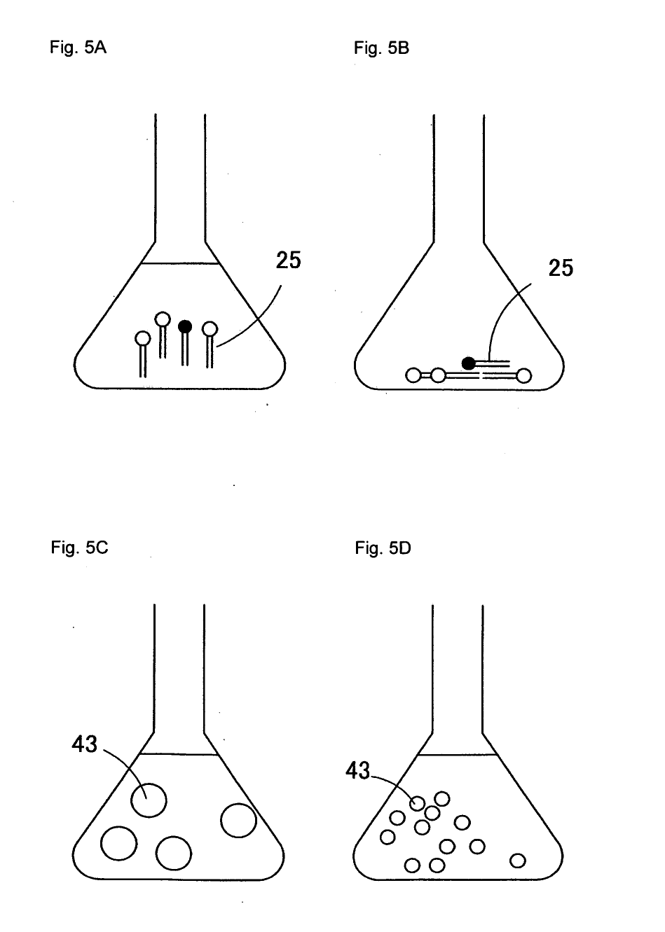

The phospholipid vesicles 43 can be prepared in the following manner. First, as shown in FIG. 5A, phospholipid 25 is fed into a flask. As the phospholipid 25, for example, 1,2-Dioleoyl-sn-Glycero-3-Phosphocholine (DOPC) with high purity can be used. The phospholipid 25 is dried in a dried Ar gas atmosphere, and is further vacuum dried for 2 hours. After the phospholipid 25 is dried as shown in FIG. 5B, water is added to to the flask to suspend the phospholipid 25 in water. Then, as shown in FIG. 5C, the suspension is ultrasonically stirred to sufficiently homogenize the phospholipid 25. Then, as shown in FIG. 5D, the homogenate is ultracentrifuged to collect supernatant, and the supernatant is stored at 4° C. This supernatant contains vesicles 43 of the phospholipid 25 having a diameter of several tens of nanometers or less.

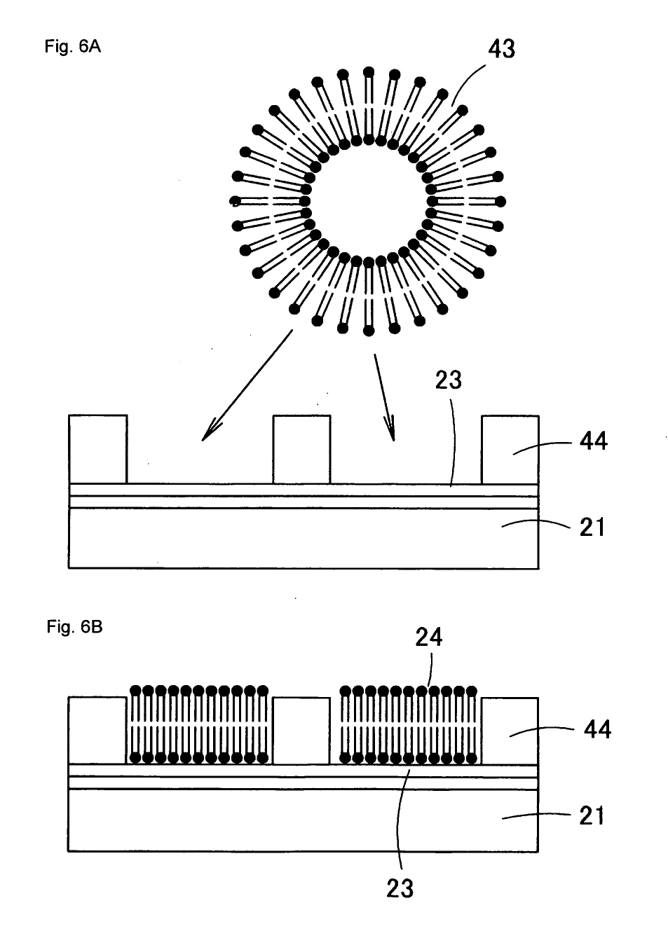

Then, as shown in FIG. 6A, the suspension containing the vesicles 43 is dropped onto a predetermined region of the monolayer 23 formed on the chip substrate 21, or the chip substrate 21 is immersed in the suspension containing the vesicles 43. By doing so, the vesicles 43 are opened due to rupture on the monolayer 23 so that lipid bilayers 24 obtained from the vesicles 43 are fused together in a chain reaction manner and self-assembled. As a result, as shown in FIG. 6B, a lipid bilayer 24 is formed on the monolayer 23 provided on the chip substrate 21. It is to be noted that in FIGS. 6A and 6B, a barrier 44 formed of a photoresist is provided on the chip substrate 21. By providing the barrier 44, it is possible to immobilize various different receptors on the lipid bilayer 24, thereby achieving a plurality of different receptor arrays.

As described above, according to the production method described above, the monolayer 23 and the lipid bilayer 24 can be easily formed on the chip substrate 21 by self-assembly, thereby enabling the substrate for immobilizing biomolecules 12 and the biochip 11 to be easily produced.

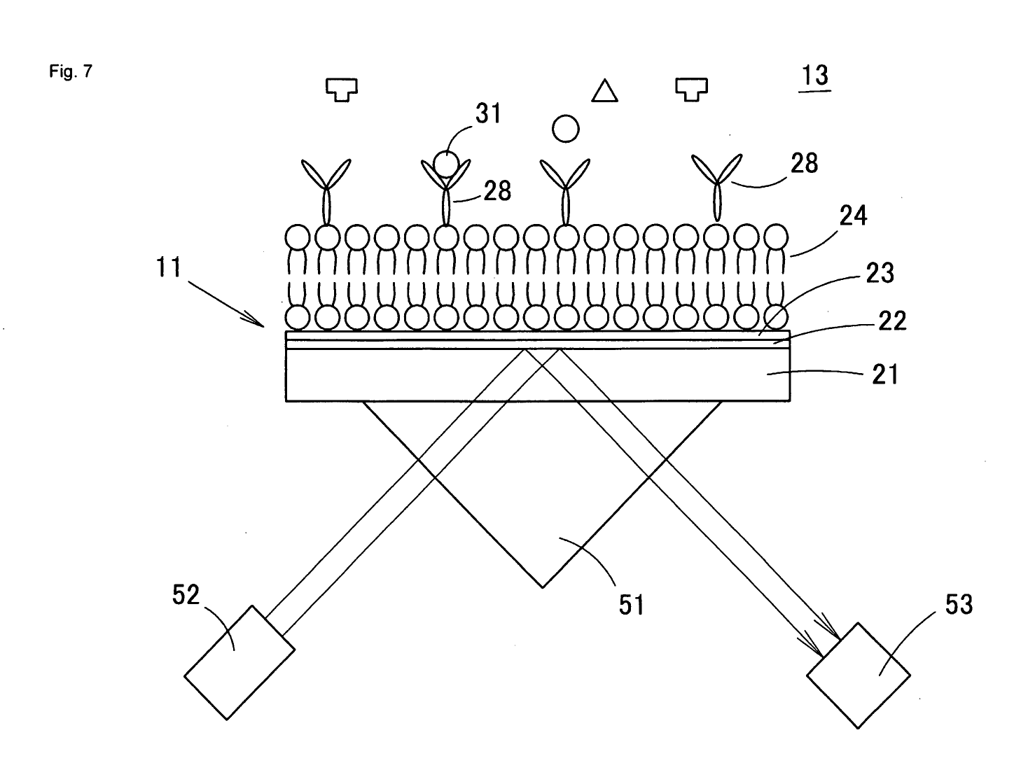

Next, a biosensor 13 using the biochip 11 according to the Example 1 will be described with reference to FIG. 7. The biosensor 13 uses surface plasmon resonance to optically detect a reaction state such as the presence or absence of an analyte 31 as a test object, the amount of the analyte 31, or the binding specificity of the analyte 31.

The biosensor 13 comprises the biochip 11 and a measuring apparatus. The measuring apparatus includes a right triangular prism 51, a light-emitting device 52, and a light-receiving device 53. The prism 51 is in close contact with the lower surface of the chip substrate 21 of the biochip 11. The light-emitting device 52 emits laser light having a visible light wavelength (e.g., 635 nm), and is arranged diagonally below the prism 51 so as to be opposite to one inclined plane of the prism 51. The light-receiving device 53 is also arranged diagonally below the prism 51 so as to be opposite to the other inclined plane of the prism 51. More specifically, the light-receiving device 53 is arranged so as to receive light emitted from the light-emitting device 52, passing through the prism 51 and the chip substrate 21, and reflected off the metal layer 22. Further, the light-emitting device 52 and the light-receiving device 53 can be moved around the prism 51. By moving the light-emitting device 52, it is possible to change the incident angle of light entering the biochip 11.

The biochip 11 is arranged in such a manner that the receptor 28 can directly come in contact with a flow path of a test sample solution. Therefore, in a case where the test sample solution contains an analyte 31 which specifically binds to the receptor 28, the analyte 31 specifically binds to the receptor 28 immobilized on the biochip 11, and is therefore immobilized on the surface of the biochip 11. When the analyte 31 is immobilized on the receptor 28, the refractive index near the metal layer 22 is changed according to the amount of the analyte 31 immobilized on the receptor 28.

As described above, the biosensor 13 uses surface plasmon resonance to detect a reaction state such as the presence or absence of the analyte 31, the amount of the analyte 31 bound to the receptor 28, or the binding specificity of the analyte 31. More specificaly, the light-emitting device 52 emits excited light in such a manner that the incident angle at an interface between the chip substrate 21 and the metal layer 22 is larger than the critical angle of total internal reflection at the interface. The excited light which has passed through the prism 51 and the chip substrate 21 is totally internally reflected off the interface between the metal layer 22 and the chip substrate 21. At this time, evanescent light is generated on the upper surface of the metal layer 22, and the electric field of the evanescent light passes through the metal layer 22 and the receptor 28 and then propagates along the upper surface of the metal layer 22.

Since the evanescent light does not propagate far from the metal layer 22 but localizes in a very small region near the upper surface of the metal layer 22, the evanescent light interacts with the analyte 31 bound to the receptor 28 but does not interact with the analyte 31 not immobilized on the receptor 28.

Therefore, reflected light received by the light-receiving device 53 is modulated according to the amount or density of the analyte 31 immobilized on the receptor 28. That is, by analyzing, for example, the reflectivity of light received by the light-receiving device 53, it is possible to measure the amount or density of a specific analyte immobilized on the receptor 28.

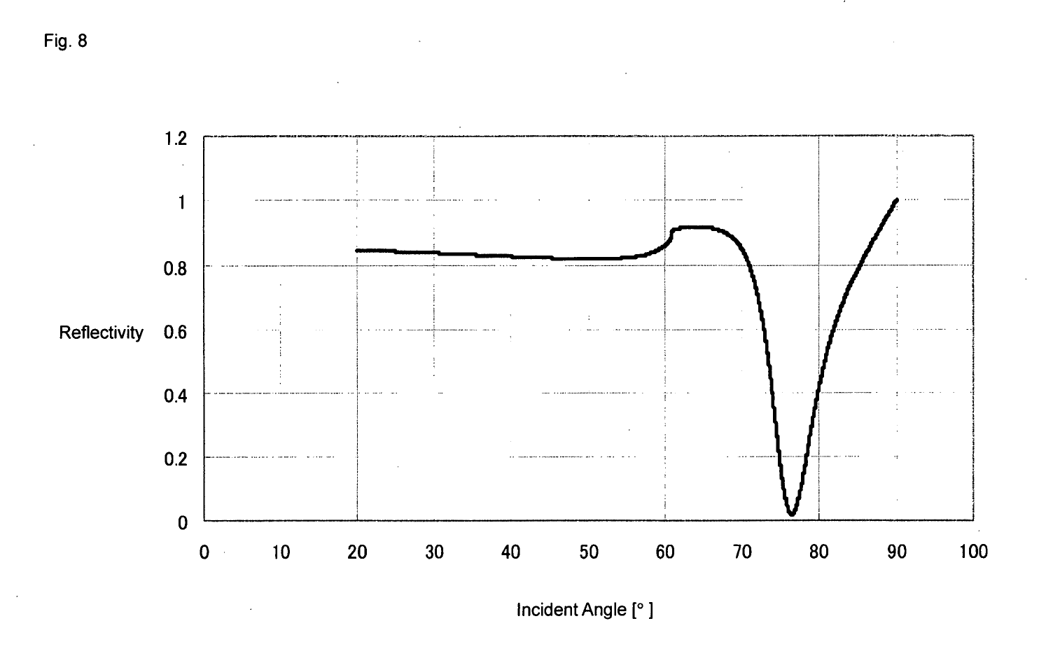

For example, when the intensity of reflected light received by the light-receiving device 53 is measured while changing the incident angle of light entering the biochip 11 by moving the light-emitting device 52, the relationship between the incident angle and reflectivity can be expressed by a curve shown in FIG. 8. Further, information about the analyte 31 can be obtained from a resonance angle (that is, an incident angle at the time when reflectivity is reduced to a minimum) and the reflectivity at the resonance angle.

As described above, since the thickness of the monolayer 23 or the lipid bilayer 24 of the biochip 11 constituting the biosensor 13 can be made uniform, the distance between the receptor 28 and the metal layer 22 can also be made uniform, thereby reducing noises and improving analytical accuracy when an analyte is analyzed by surface plasmon resonance. Further, since the thickness of the monolayer 23 can be controlled with angstrom (Å) accuracy, the thickness of the monolayer 23 can be adjusted (especially, the thickness of the monolayer can be decreased) so that the receptor and the analyte can be located at a position where the sensing sensitivity of the biosensor 13 is enhanced. This makes it possible to produce a biosensor 13 having a good S/N ratio.

Such a biosensor can be used for various medical purposes such as physical examination and checking the presence or absence of pathogen in blood, and for other purposes such as food inspection (e.g., checking the kinds of proteins contained in foods) and environmental measurement. Further, the biosensor can also be used for purposes of security and person authentication by checking an analyte specific to an individual.

Further, the monolayer 23 and the lipid bilayer 24 of the biochip 11 can be dissociated from each other using a surfactant. For example, when a used biochip 11 is immersed in an SDS solution (SDS: Sodium dodecyl sulfate, H₃C—(CH₂)₁₀—CH₂OSO₃—Na+) as a surfactant, the lipid bilayer 24 is dissociated from the monolayer 23. In this way, the lipid bilayer 24 is easily removed from a used biochip 11. Therefore, it becomes possible to form a new lipid bilayer 24 on the monolayer 23, thereby allowing regeneration and reuse of the biochip 11.

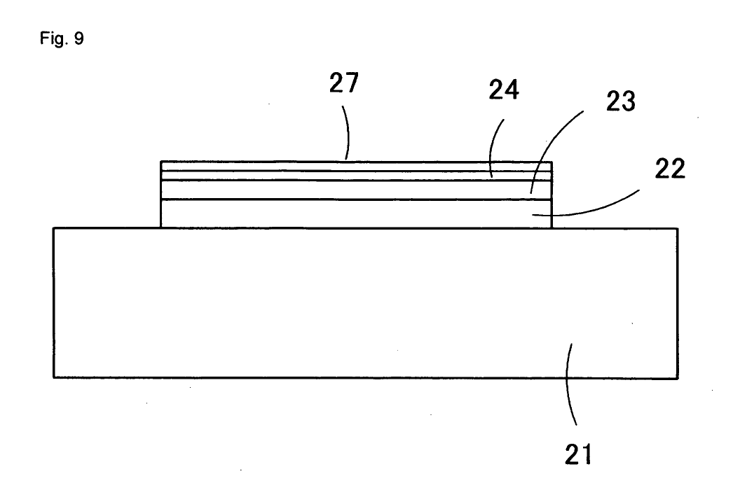

Finally, the results of simulating the performance of biosensor according to an embodiment of the present invention will be described. FIG. 9 shows a schematic view of a model used for simulation. A chip substrate 21 is a transparent substrate having a refractive index of 1.52. A metal layer 22 is an Au layer having a thickness of 50 nm. A monolayer 23 has a refractive index of 1.5 and a thickness of 2 nm. A lipid bilayer 24 has a refractive index of 1.49 and a thickness of 5 nm. A layer of a biorecognition molecule 27 has a refractive index of 1.57 and a thickness of 10 nm. A sample solution containing an analyte had a refractive index of 1.33.

Changes in resonance angle and reflectivity at the time when the thickness of the monolayer 23 was changed in the range of 0.1 nm to 2 nm were determined using the model. Further, changes in resonance angle and reflectivity at the time when the thickness of the lipid bilayer 24 was changed in the range of 5 nm to 10 nm were determined using the model. Furthermore, changes in resonance angle and reflectivity at the time when the thickness of the metal layer 22 was changed in the range of 30 nm to 80 nm were determined using the model. In this regard, it is to be noted that the wavelength of incident light was 635 nm, and the incident angle of the incident light was changed in the range of 20° to 90°.

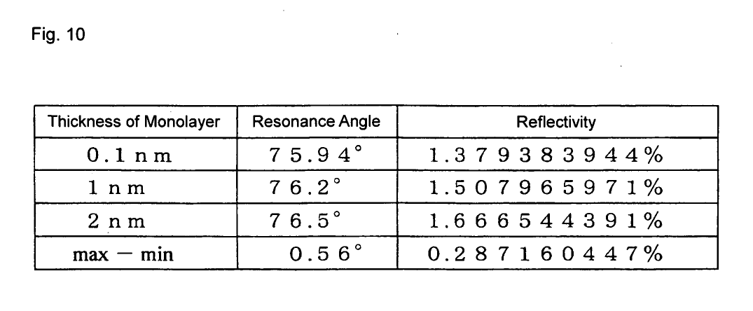

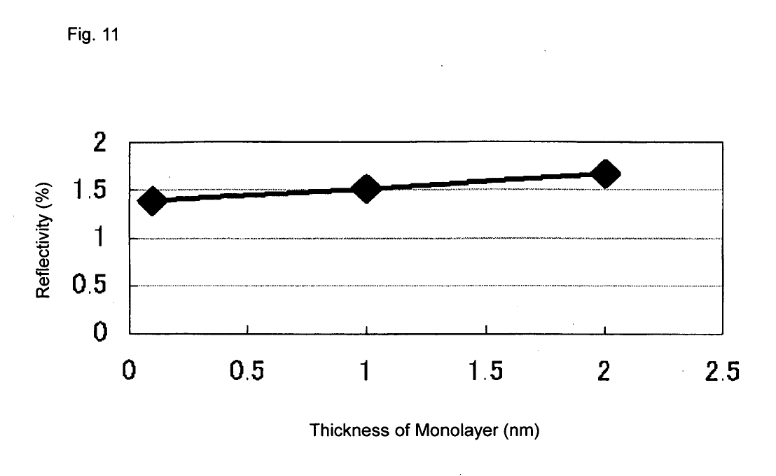

FIG. 10 shows a result of determining changes in resonance angle and reflectivity at the time when the thickness of the monolayer 23 was changed (2 nm, 1 nm, and 0.1 nm). FIG. 11 shows a graph obtained by plotting the values listed in FIG. 10 to illustrate a change in reflectivity. As can be seen from the result, the smaller the thickness of the monolayer 23, the smaller the resonance angle and the reflectivity. Particularly, the reflectivity varies linearly with the thickness of the monolayer 23. Since a smaller reflectivity improves analytical accuracy the thickness of the monolayer 23 is kept as small as possible.

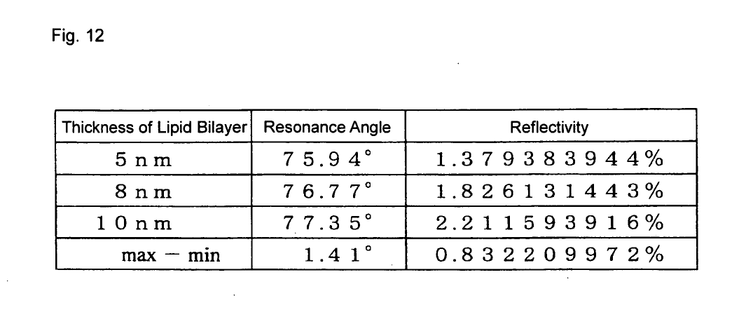

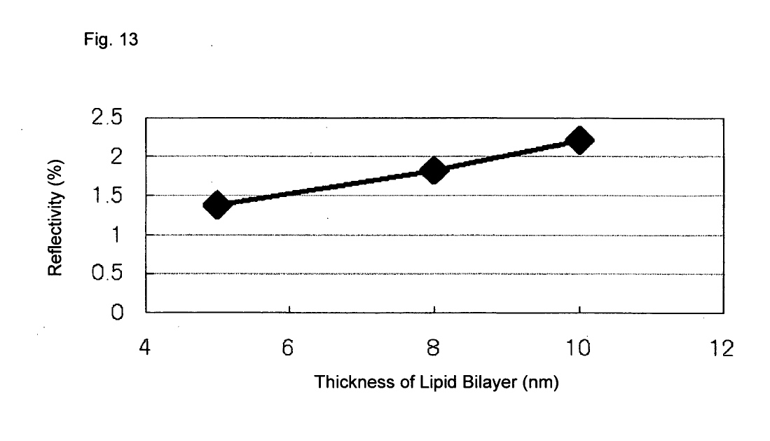

FIG. 12 shows a result of determining changes in resonance angle and reflectivity at the time when the thickness of the lipid bilayer 24 was changed (10 nm, 8 nm, and 5 nm). FIG. 13 shows a graph obtained by plotting the values listed in FIG. 12 to illustrate a change in reflectivity. As can be seen from the result, the smaller the thickness of the lipid bilayer 24, the smaller the resonance angle and the reflectivity. Particularly, the reflectivity varies linearly with the thickness of the lipid bilayer 24. Since a smaller reflectivity improves analytical accuracy, the thickness of the lipid bilayer 24 is kept as small as possible.

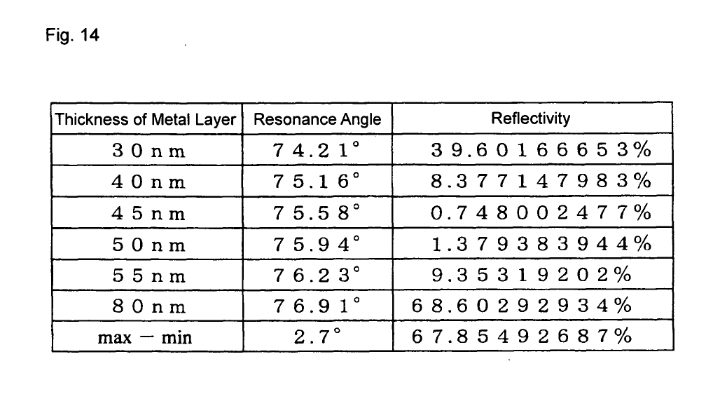

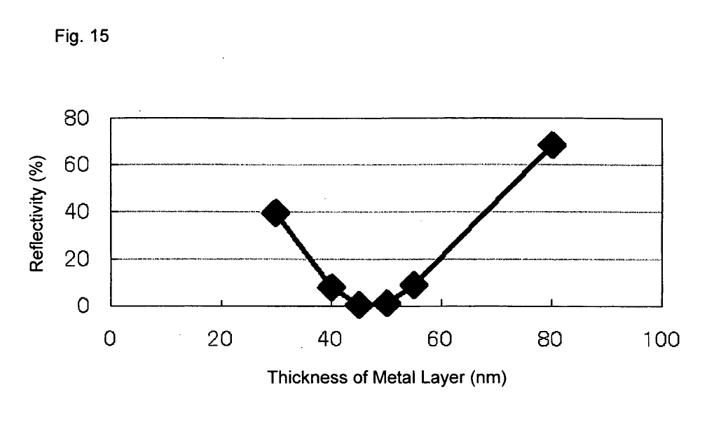

FIG. 14 shows a result of determining changes in resonance angle and reflectivity at the time when the thickness of the metal layer 22 was changed (80 nm, 55 nm, 50 nm, 45 nm, 40 nm, and 30 nm). FIG. 15 shows a graph obtained by plotting the values listed in FIG. 14 to illustrate a change in reflectivity. As can be seen from the result, the smaller the thickness of the metal layer 22, the smaller the resonance angle. On the other hand, as can be seen from FIG. 15, the reflectivity exhibits a minimum when the thickness of the metal layer 22 is in the range of 30 nm to 80 nm. This indicates that an optimum thickness exists for the metal layer 22 (in this simulation, an optimum thickness of the metal layer 22 is about 45 nm). Therefore in several embodiments, the metal layer 22 has a thickness close to such an optimum thickness.

What is claimed is:

1. A substrate for immobilizing biomolecules comprising:
a substrate;
anchoring molecules provided on the substrate; and
a lipid bilayer provided on the anchoring molecules,
wherein the anchoring molecules are represented by X—(CH₂)n-OH (where X is a thiol group) and form a layer; and the lipid bilayer is anchored to the substrate via hydrogen bonds existing between the lipid bilayer and the anchoring molecules.

2. The substrate for immobilizing biomolecules according to claim 1, wherein the density of the anchoring molecules forming the layer is 1 molecule/nm² or more.

3. The substrate for immobilizing biomolecules according to claim 1, further comprising a thin layer of an inorganic material such as Au or Ag provided on the substrate.

4. The substrate for immobilizing biomolecules according to claim 1, wherein the lipid bilayer can be dissociated from the layer formed by the anchoring molecules.

5. A biochip comprising:
a substrate;
anchoring molecules provided on the substrate;
a lipid bilayer provided on the anchoring molecules;
a biorecognition molecule immobilized on the lipid bilayer; and
a receptor immobilized on the biorecognition molecule,
wherein the anchoring molecules are represented by X—(CH₂)n-OH (where X is a thiol group) and form a layer; the lipid bilayer is anchored to the substrate via hydrogen bonds existing between the lipid bilayer and the anchoring molecules; and the receptor specifically binds to a specific protein (ligand).

6. The biochip according to claim 5, wherein the biorecognition molecule comprises biotin immobilized on the lipid biolayer; and avidin, and the receptor is an antibody labeled with biotin.

7. A biosensor comprising:
a biochip; and
a measuring apparatus,
wherein the biochip comprises a substrate; anchoring molecules provided on the substrate; a lipid bilayer provided on the anchoring molecules; a biorecognition molecule immobilized on the lipid bilayer; and a receptor immobilized on the biorecognition molecule, wherein the anchoring molecules are represented by X—(CH₂)ₙ—OH (where X is a thiol group) and form a layer; the lipid bilayer is anchored to the substrate via hydrogen bonds existing between the lipid bilayer and the anchoring molecules; and the receptor specifically binds to a specific protein (ligand), and wherein the measuring apparatus detects a reaction state such as the presence or absence of an analyte as a test object, the amount of the analyte, or the binding specificity of the analyte.

8. The biosensor according to claim 7, wherein the measuring apparatus uses surface plasmon resonance (SPR).

9. The biosensor according to claim 7, further comprising an Au thin layer provided on the surface of the substrate of the biochip,
wherein the thickness of the Au thin layer or the diameter of an Au particle is 40 nm or more but 50 nm or less; the thickness of the layer formed by the anchoring molecules is 1 nm or less; the thickness of the lipid bilayer is 5 nm or more but 10 nm or less; and the wavelength of light to be used for surface plasmon resonance is a visible light wavelength.

10. A method for forming a substrate to which a lipid bilayer is anchored comprising the steps of:
forming a layer by arranging anchoring molecules represented by X—(CH₂)n-OH (where X is a thiol group) on the surface of a substrate by self-assembly; and
forming on the layer formed by the anchoring molecules, a lipid bilayer by lipid self-assembly and anchoring the lipid bilayer to the substrate via hydrogen bonds existing between the lipid bilayer and the anchoring molecules.
